(19) United States
(12) Patent Application Publication
(10) Pub. No.: US 2018/0311215 A1
(43) Pub. Date: Nov. 1, 2018

(54) PHARMACEUTICAL COMPOSITION AND PREPARATION METHOD THEREFOR

(71) Applicants: HANGZHOU MINSHENG INSTITUTE FOR PHARMA RESEARCH CO., LTD., Hangzhou, Zhejiang (CN); HANGZHOU MINSHENG PHARMACEUTICAL CO., LTD., Hangzhou, Zhejiang (CN)

(72) Inventors: Weiliang XU, Hangzhou, Zhejiang (CN); Jingya FANG, Hangzhou, Zhejiang (CN); Feiyu FENG, Hangzhou, Zhejiang (CN); Lan YANG, Hangzhou, Zhejiang (CN); Dianwu GUO, Hangzhou, Zhejiang (CN); Fujiang ZHU, Hangzhou, Zhejiang (CN); Qiufu GE, Hangzhou, Zhejiang (CN)

(73) Assignee: Hangzhou TINO Pharma Co., Ltd., Hangzhou, Zhejiang (CN)

(21) Appl. No.: 15/770,138

(22) PCT Filed: Oct. 20, 2016
(86) PCT No.: PCT/CN2016/102701

§ 371 Date: Apr. 20, 2018

(30) Foreign Application Priority Data
Oct. 20, 2015 (CN) 201510684182.3

(51) Int. Cl. A61K 31/4184; A61K 9/00; A61K 9/19

(52) U.S. Cl. CPC A61K 31/4184; A61K 9/19; A61K 9/0019

(57) ABSTRACT

The invention provides a pharmaceutical composition for injection comprising a NL-101 type compound and a preparation method thereof. The composition uses the NL-101 type compound as an active pharmaceutical ingredient, and the stability of the NL-101 type compound is improved by adding a chloride-containing stabilizing agent or using a co-solvent containing hydrochloric acid, so that the formulation can be stably produced and meet the requirements for clinical safe use.

### FIELD OF THE INVENTION

The present invention belongs to the art of medicine and particularly relates to an injection drug composition containing NL-101 compound and a preparation method thereof.

### BACKGROUND OF THE INVENTION

Bendamustine, a well-known chemotherapeutic drug that was first synthesized in 1963, consists of an alkylated mechlorethamine moiety and a benzimidazole moiety that functions as a purine analogue (Barman Balfour J A et al., Drugs, 2001; 61: 631-640). Bendamustine has excellent activity in the following cancers: low-grade lymphoma (Herold M et al., Blood, 1999; 94, Suppl. 1: 262a), multiple myeloma (Poenisch W et al., Blood, 2000; 96, Suppl 1: 759a) and several solid tumors (Kollmannsberger C et al., Anticancer Drugs, 2000; 11: 535-539). Bendamustine is also reported to be effective in inducing apoptosis in lymphoma cells (Chow K U et al., Haematologica, 2001; 86: 485-493). In March 2008, the FDA approved bendamustine for the treatment of chronic lymphocytic leukemia (CLL). In October 2008, the FDA approved bendamustine for the treatment of indolent B-cell non-Hodgkin's lymphoma (NHL) that has progressed during or within six months of treatment with rituximab or a regimen containing rituximab.

The clinical activity of bendamustine as a single drug and in combination with other chemotherapy drugs and immunotherapy drugs, and its possible lack of cross-resistance with many other chemotherapeutic agents make bendamustine an excellent choice for patients with newly diagnosed and refractory hematological malignancies (Leoni L M, Semin Hematol. April 2011; 48 Supplement 1: S4-11). Currently, approximately 75 clinical trials for bendamustine in various cancer indications such as leukemia, lymphoma, small cell lung cancer, multiple myeloma, MDS, ovarian cancer, breast cancer, and brain tumors are in progress.

In recent years, histone deacetylase (HDAC) is becoming an important disease target for cancer treatment (Minucci, S. et al., Nat Rev Cancer, 2006, 6, 38-51). According to their sequence homology, human HDAC enzymes are divided into class I-IV of 18 subtypes. Classes I, II, and IV generally refer to classical HDACs and consist of 11 family members. Class III HDACs include seven enzymes, and because of their distinct differences from other HDAC family members, they have a unique term, Sirtuins. Inhibition of HDAC enzymes can lead to histone acetylation, which is related to chromatin remodeling and plays an important role in the epigenetic regulation of gene expression. In addition, studies have shown that HDAC inhibitors can also cause the acetylation of many important non-histone enzymes, such as HSP90, α-tubulin, Ku-70, Bcl-6, importin, cortical actin, P53, STAT1, E2F1, GATA-1 and NF-kB, and acetylation of these enzymes will alter many important signaling pathways associated with cancer treatment. The antitumor mechanisms of HDAC inhibitors include cell differentiation, cell cycle arrest, inhibition of DNA repair, induction of apoptosis, upregulation of tumor suppressor genes, downregulation of growth factors, oxidative stress, and autophagy. Over the past decade, many HDAC inhibitors different in structures have been discovered, of which at least 12 HDAC inhibitors have entered clinical trials for cancer therapy, including short-chain fatty acids (valproic acid), hydroxamic acids (SAHA, LBH589, PXD101, JNJ-26481585, ITF2357, CUDC-101), cyclic tetrapeptide (FK-228), benzamide (MS-275), and several other compounds (CHR-3996, 4SC-201, SB939).

NL-101 is a bifunctional bendamustine derivative that potently inhibits the HDAC pathway. The chemical structure, molecular formula, and molecular weight of NL-101 are shown below, and its chemical name is 7-[5-[bis(chloroethyl)-amino]-1-methylbenzimidazol-2-yl]-N-hydroxyl-heptamide.

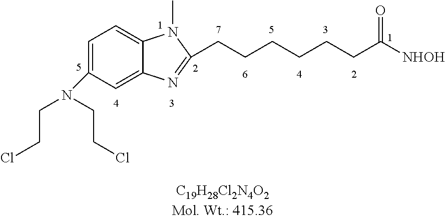

NL-101 is the world's first anti-cancer compound with both DNA damage and histone deacetylase (HDAC) inhibitory activity, which was first disclosed in WO2010085377A2, whose Chinese family patent CN102186842B has been granted, and the patentees are Hangzhou Minsheng Pharmaceutical Co., Ltd. and Northlake Biosciences LLC, the full text of which is incorporated herein by reference.

NL-101 is a white to almost white crystalline powder, light weight, insoluble in water, very slightly soluble in ethanol, slightly soluble in methanol, and easily soluble in acetic acid. In case of strong acid, strong alkali, strong oxidant and light, it is easily degraded, unstable in aqueous solution, and relatively stable in methanol and acetic acid solution.

The NL-101 molecule contains a bis(chloroethyl)amino group and a hydroxamic acid group, making it highly chemically active (easily hydrolyzed) in an aqueous solution and having poor stability.

Regarding the NL-101 pharmaceutical preparation, in the prior art, there is an injection solution that NL-101 is directly dissolved with acetic acid and then diluted with water for injection. Animal experiment shows that the intravenous injection of the injection is highly toxic, and the animal has a high mortality rate and a large vascular irritation.

Northlake Biosciences LLC submitted an application WO2013010286A2 on Sep. 14, 2012, and its Chinese family patent is CN103826630A. Chinese family patent application CN103826630A uses acetic acid to dissolve NL-101 and use cyclodextrin inclusion technology to prepare lyophilized powder for injection to improve the safety of NL-101. However, the prepared NL-101 lyophilized powder for injection, such as Example 2, although claimed to be chemically stable at −20° C., 4° C. and room temperature for at least 2 weeks, in fact, the inventors of the present application have verified that the results are still far from satisfying the clinical application requirements of drugs. Although it can be stable for 2-3 weeks at −20° C. and 4° C., it is only stable for several days at room temperature. In addition, it was also found that the intermediate solution before lyophilization obtained according to Example 2 of the application was also very poor in stability, and when it was left in the dark at room temperature for 4 hours, more than 15% of NL-101 was degraded. The lyophilized powder for injection was left at room temperature for 1 month and NL-101 was degraded severely. Especially when placed only 5 days in the condition of high temperature accelerated test at 40° C., more than 15% of NL-101 was degraded. The specific stability test results are shown in Table 1, Table 2, and Table 3 below.

TABLE 1
Stability test results for NL-101 intermediate solution
placed at room temperature (25 ± 2° C.)

| Test | intermediate | intermediate | intermediate |
|---|---|---|---|
| Item | solution at 0 h | solution at 2 h | solution at 4 h |
| Purity | 94.05% | 88.06% | 78.22% |

TABLE 2
Stability test results for lyophilized preparations of NL-101
placed at room temperature (25 ± 2° C.)

| | lyophilized | lyophilized | lyophilized | lyophilized |
|---|---|---|---|---|
| Test | preparations | preparations | preparations | preparations |
| Item | at day 0 | at day 5 | at day 15 | at day 30 |
| Purity | 90.23% | 85.16% | 77.31% | 70.55% |

TABLE 3
Stability test results for lyophilized preparations
of NL-101 placed in the accelerated test at 40° C.

| | lyophilized | lyophilized |
|---|---|---|
| Test | preparations | preparations |
| Item | at day 0 | at day 5 |
| Purity | 90.23% | 72.31% |

Obviously, the NL-101 lyophilized preparation for injection prepared according to the prior art has poor stability and cannot meet the requirements for clinical stability (including storage, transportation, sales, and use) of the drug.

### SUMMARY OF THE INVENTION

The purpose of the present invention is to overcome the shortcomings of the existing NL-101 compound preparations and to provide a stable NL-101 pharmaceutical preparation that can be stably produced and can meet the requirements for clinical safe use.

In the process of investigating the NL-101 lyophilized preparation, the inventors unexpectedly discovered that reconstitution of the NL-101 lyophilized preparation with different solvents has a great influence on the stability of NL-101. For example, the lyophilized preparation prepared in Example 2 of CN103826630A was reconstituted with water for injection and physiological saline, respectively, and the purity of NL-101 in the reconstituted solution was examined at different time points. It was found that the stability of the reconstituted solution using physiological saline was significantly better than that using water for injection, and the results are shown in Table 4 below.

TABLE 4
Stability test results for NL-101 reconstituted solution (5 mg/mL)
placed at room temperature (25 ± 2° C.)

| Item | 0 h | 2 h | 4 h | 6 h |
|---|---|---|---|---|
| purity of NL-101 reconstituted | 90.23% | 80.06% | 71.08% | 59.36% |
| with water for injection | | | | |
| purity of NL-101 reconstituted | 91.88% | 87.05% | 80.23% | 78.33% |
| with sodium chloride injection | | | | |

Analysis of the results in Table 4 shows that the sodium chloride in the reconstituted solution may increase the stability of NL-101. Since the NL-101 lyophilized powder for injection prepared in Example 2 of CN103826630A contains sodium ions, it is speculated that the stability of NL-101 is increased by chlorine ions in sodium chloride. In order to confirm this, we conducted a stability investigation by reconstituting the lyophilized preparation prepared in Example 2 of CN103826630A in a potassium chloride solution having the same molar concentration as physiological saline. The results showed that the reconstituted solution using potassium chloride was also more stable than the reconstituted solution using water for injection, and its stability was similar to that of the reconstituted solution using physiological saline. The results are shown in Table 5 below.

TABLE 5
Stability test results for NL-101 reconstituted solution (5 mg/mL)
placed at room temperature (25 ± 2° C.)

| Item | 0 h | 2 h | 4 h | 6 h |
|---|---|---|---|---|
| purity of NL-101 reconstituted | 91.28% | 86.25% | 81.93% | 83.56% |
| with potassium chloride injection | | | | |

Based on this, we believe that the introduction of chlorine ions in the preparation of NL-101 can increase the stability of NL-101. On this basis, the present invention has been completed.

According to one aspect of the present invention, the present invention provides a pharmaceutical composition comprising a compound of formula (I) or a pharmaceutically acceptable salt thereof and a stabilizing agent, and the stabilizing agent is a pharmaceutically acceptable chloride-containing compound,

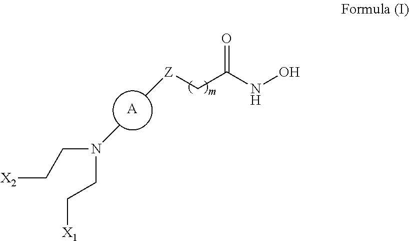

Wherein,

m is an integer selected from 5-16;

Z is absent or selected from C(RaRb), O, S, C(O), N(Ra), SO₂, OC(O), C(O)O, OSO₂, S(O₂)O, C(O)S, SC(O), C(O)C(O), C(O)N(Ra), N(Ra)C(O), S(O₂)N(Ra), N(Ra)S(O₂), OC(O)N(Ra), N(Ra)C(O)O, N(Ra)C(O)S, or N(Ra)C(O)N(Rb), wherein Ra and Rb are each independently H, alkyl, alkenyl or alkynyl;

X₁ and X₂ are each independently halogen or OSO₂Rc, wherein Rc is alkyl, alkenyl or alkynyl;

Q is selected from cycloalkyl, heterocycloalkyl, cycloalkenyl, heterocycloalkenyl, aryl or heteroaryl, optionally substituted with one or more substituents selected from alkyl, alkenyl, alkynyl, cycloalkyl, heterocycloalkyl, cycloalkenyl, heterocycloalkenyl, aryl, heteroaryl, halogen, nitro, oxo, —C═NH, cyano, alkyl-Rd, ORd, OC(O)Rd, OC(O)ORd, OC(O)SRd, SRd, C(O)Rd, C(O)ORd, C(O)SRd, C(O)NReRf, SORd, SO₂Rd, NReRf, or N(Re)C(O)Rf, wherein Rd, Re, and Rf are each independently selected from H, alkyl, alkenyl, alkynyl, cycloalkyl, heterocycloalkyl, aryl, heteroaryl, halogen, cyano, amine, nitro, hydroxyl, or alkoxy.

In the preferred embodiment of the present invention,

m is selected from 5, 6, 7 or 8;

Z is absent or selected from CH₂, O, CO, NH, SO₂, OC(O), C(O)O, C(O)S, NHC(O), C(O)NH, OC(O)NH, NHC(O)O, or NHC(O)S;

X₁ and X₂ are independently halogen; and

Q is 9-10 membered aryl or heteroaryl.

More preferably, Z is absent or selected from CH₂, O, CO, NH, SO₂, NHC(O), or C(O)NH.

In the more preferred embodiment of the present invention, the compound of formula (I) is represented by the following formula (II):

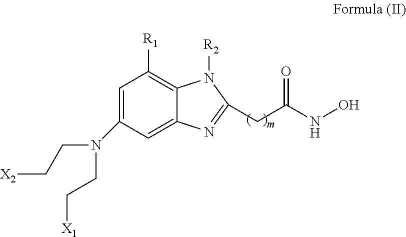

wherein R₁ and R₂ are each independently selected from H, alkyl, alkenyl, alkynyl, cycloalkyl, heterocycloalkyl, halogen, —C═NH, amine, cyano, hydroxy, or alkoxy.

In the most preferred embodiment of the present invention, the compound of formula (I) is selected from NL-101 as represented by the following formula:

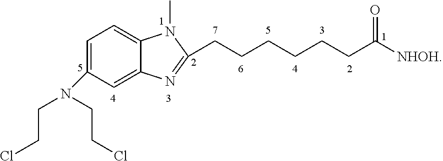

That is, the present invention provides a pharmaceutical composition comprising NL-101 or a pharmaceutically acceptable salt thereof, and a stabilizing agent, and the stabilizing agent is a pharmaceutically acceptable chloride-containing compound.

In the present invention, a compound of formula (I) (preferably a compound of formula (II), more preferably NL-101) can be reacted with an inorganic or organic acid to form a pharmaceutically acceptable acid addition salt or reacted with an inorganic or organic base to form a pharmaceutically acceptable base addition salt.

For example, hydrochloride, hydrobromide, hydroiodide, sulfate, dodecyl sulfate, hydrogen sulfate, bisulfate, hemisulfate, persulfate, bicarbonate, carbonate, phosphate, metaphosphate, monohydrogen phosphate, dihydrogen phosphate, glycerophosphate, nitrate, methane sulfonate, benzene sulfonate, tosylate, mesylate, ethane sulfonate, 2-hydroxyethane sulfonate, isethionate, 2-naphthalenesulfonate, benzoate, phenylacetate, chlorobenzoate, dinitrobenzoate, acetate, butyrate, isobutyrate, hexanoate, octanoate, heptanoate, cypionate, malonate, succinate, adipate, pamoate, phthalate, fumarate, maleate, lactate, galactosylate, lacturate, citrate, tartrate, maleate, succinate, hemisuccinate, nicotinate, oxalate, oleate, salicylate, ascorbate, mandelate, alginate, fumarate, mucrate[hhl1], hippurate, gluconate, digluconate, pectate, glutamate, arginine, aspartate, histidine salt, lysine salt, camphor salt, camphor sulfonate, phosphonate, sodium hydroxide salt, potassium hydroxide salt, lithium hydroxide salt, barium hydroxide salt, calcium hydroxide salt, potassium ethoxide salt, n-propanol sodium salt, ammonium hydroxide salt, piperidine salt, N-ethylpiperidine salt, piperazine salt, morpholine salt, N-ethylmorpholine salt, ethanolamine salt, diethanolamine salt, triethanolamine salt, ethylenediamine salt, hydroxylamine salt, isopropylamine salt, dicyclohexylamine salt, trimethylamine salt, triethylamine salt, tripropylamine salt, meglumine salt, glucosamine salt, N-methyl-D-glucosamine salt, N,N′-dibenzylethylenediamine salt, tromethamine salt, 2-diethylaminoethanol, 2-dimethylaminoethanol, N-methylglutamine salt, tromethamine salt, Betaine salt, caffeine salt, procaine salt, chloroprocaine salt, lidocaine salt, choline salt, purine salt, theobromine salt, methyl methyl benzoate, polyamine resin, etc., preferably hydrochloride, methane sulfonate, tosylate, acetate, succinate, citrate, maleate, tartrate.

Those skilled in the art can understand that the pharmaceutical composition of the present invention can be made into various dosage forms.

In a preferred embodiment, the pharmaceutical composition of the present invention may further comprise a co-solvent, a lyophilizing protectant, a pH controlling agent, and water for injection.

In a preferred embodiment, the stabilizing agent is a mixture selected from one or more of sodium chloride, potassium chloride, and hydrochloric acid, and preferably is sodium chloride.

The co-solvent is a pharmaceutically acceptable acidic solvent, preferably acetic acid and/or citric acid.

The lyophilizing protectant is a pharmaceutically acceptable cyclic polysaccharide or a mixture thereof, preferably the cyclic polysaccharide is cyclodextrin, cyclomannin, cycloaltrin, cyclofructin or an analog thereof, more preferably the cyclic polysaccharide is cyclodextrin or a derivative thereof, still more preferably the cyclic polysaccharide is α-cyclodextrin or a derivative thereof, β-cyclodextrin or a derivative thereof, or γ-cyclodextrin or a derivative thereof, further preferably the cyclic polysaccharide is β-cyclodextrin or a derivative thereof, most preferably the cyclic polysaccharide is β-cyclodextrin, sulfobutylether-β-cyclodextrin and/or hydroxypropyl-β-cyclodextrin.

The pH controlling agent is a mixture selected from one or more of sodium hydroxide, sodium carbonate, potassium carbonate, sodium hydrogencarbonate, potassium hydrogencarbonate, sodium citrate, and potassium citrate, and preferably is sodium hydroxide.

In the present invention, the compound of the formula (I) or the compound of the formula (II) or NL-101 or a pharmaceutically acceptable salt thereof has a mass volume percentage concentration of 0.1-5.0%, preferably 0.2-2.0%, further preferably 0.5-1.0%, still further preferably 0.5%. The stabilizing agent has a mass volume percentage concentration of 0.4-10.0%, preferably 0.9-8.0%, further preferably 1.0-7.0%, still further preferably 2.0-6.0%, still further preferably 3.0-5.0%. The co-solvent has a mass volume percentage concentration of 0.5-25.0%, preferably 1.0-20.0%, further preferably 1.0-10.0%, still further preferably 1.25-5.0%. The lyophilizing protectant has a mass volume percentage concentration of 2.0-35.0%, preferably 5.0-30.0%, still further preferably 10.0-20.0%. The pH of the composition is 3.0-7.0, preferably 4.0-6.0, further preferably 5.0.

According to another aspect of the present invention, the present invention also provides a lyophilized pharmaceutical composition for injection, which is prepared by lyophilizing the above-described pharmaceutical composition.

According to another aspect of the present invention, the present invention further provides a method for preparing the above-described lyophilized pharmaceutical composition for injection, comprising the steps of:

A. the lyophilizing protectant and stabilizing agent are weighed and dissolved in a suitable amount of water for injection to give solution 1;

B. the compound of formula (I) (preferably compound of formula (II), more preferably NL-101) or a pharmaceutically acceptable salt thereof is weighed and dissolved with a co-solvent to give a concentrated solution which is then added to the solution 1, the pH controlling agent is used to adjust the pH, and finally the volume is adjusted with water for injection to obtain diluted solution;

C. the diluted solution is filtrated, sterilized and filled in, half-stoppered, and put into a lyophilizer;

D. following lyophilization, taken out of the lyophilizer, fully stoppered and capped to give the pharmaceutical composition.

In a preferred embodiment, the co-solvent is firstly formulated with water for injection as a solution of co-solvent. When the co-solvent is acetic acid or citric acid, preferably the volume percent concentration of acetic acid or citric acid in the solution of the co-solvent is 30-100%, further preferably 40-80%, still further preferably 40-60%, and still further preferably 50%.

In the present invention, when hydrochloric acid is used as a co-solvent, since chlorine ions are introduced, a stabilizing agent may not be used at this time because hydrochloric acid has the function of a stabilizing agent at the same time. Therefore, according to another aspect of the present invention, the present invention further provides the following invention content:

A pharmaceutical composition comprising a compound of formula (I) (preferably a compound of formula (II), more preferably NL-101) or a pharmaceutically acceptable salt thereof and a co-solvent, and the co-solvent is hydrochloric acid, or a mixture of hydrochloric acid with a pharmaceutically acceptable acidic solvent. Preferably, the co-solvent is hydrochloric acid, or a mixture of hydrochloric acid with a pharmaceutically acceptable acidic solvent; more preferably, the co-solvent is hydrochloric acid, or a mixture of hydrochloric acid with acetic acid and/or citric acid. When the co-solvent is a mixture of hydrochloric acid with a pharmaceutically acceptable solvent (preferably an acidic solvent, more preferably acetic acid and/or citric acid), the volume percentage of hydrochloric acid and the pharmaceutically acceptable solvent may be any range, preferably, the volume percentage of hydrochloric acid and pharmaceutically acceptable solvent is 1:99 to 99:1; more preferably 5:95 to 95:5; still more preferably 10:90 to 90:10; and also 20:80 to 80:20; for example, 30:70 to 70:30.

Those skilled in the art can understand that the pharmaceutical composition of the present invention can be made into various dosage forms.

In a preferred embodiment, the pharmaceutical composition of the present invention may further comprise a lyophilizing protectant, a pH controlling agent, and water for injection.

In a preferred embodiment, the lyophilizing protectant is a pharmaceutically acceptable cyclic polysaccharide or a mixture thereof, preferably the cyclic polysaccharide is cyclodextrin, cyclomannin, cycloaltrin, cyclofructin or an analog thereof, more preferably the cyclic polysaccharide is cyclodextrin or a derivative thereof, still more preferably the cyclic polysaccharide is α-cyclodextrin or a derivative thereof, β-cyclodextrin or a derivative thereof, or γ-cyclodextrin or a derivative thereof, further preferably the cyclic polysaccharide is β-cyclodextrin or a derivative thereof, most preferably the cyclic polysaccharide is β-cyclodextrin, sulfobutylether-β-cyclodextrin and/or hydroxypropyl-β-cyclodextrin.

The pH controlling agent is a mixture selected from one or more of sodium hydroxide, sodium carbonate, potassium carbonate, sodium hydrogencarbonate, potassium hydrogencarbonate, sodium citrate, and potassium citrate, and preferably is sodium hydroxide.

In a preferred embodiment, the pharmaceutical composition of the present invention may further comprise a stabilizing agent. Preferably, the stabilizing agent is a mixture selected from one or more of sodium chloride, potassium chloride, and hydrochloric acid, and further preferably is sodium chloride.

When the stabilizing agent is selected from hydrochloric acid, the total amount of hydrochloric acid added is the sum of the amount of the co-solvent added and the amount of the stabilizing agent added.

In the present invention, the compound of the formula (I) or the compound of the formula (II) or NL-101 or a pharmaceutically acceptable salt thereof has a mass volume percentage concentration of 0.1-5.0%, preferably 0.2-2.0%, further preferably 0.5-1.0%, still further preferably 0.5%. The co-solvent has a mass volume percentage concentration of 0.5-25.0%, preferably 1.0-20.0%, further preferably 1.0-10.0%, still further preferably 1.25-5.0%. The lyophilizing protectant has a mass volume percentage concentration of 2.0-35.0%, preferably 5.0-30.0%, further preferably 10.0-20.0%. The stabilizing agent has a mass volume percentage concentration of 0-10.0%, preferably 0.9-8.0%, further preferably 1.0-7.0%, still further preferably 2.0-6.0%, still further preferably 3.0-5.0%. The pH of the composition is 3.0-7.0, preferably 4.0-6.0, further preferably 5.0.

According to another aspect of the present invention, the present invention also provides a lyophilized pharmaceutical composition for injection, which is prepared by lyophilizing the above-described pharmaceutical composition.

According to another aspect of the present invention, the present invention further provides a method for preparing the above-described lyophilized pharmaceutical composition for injection, comprising the steps of:

A. the lyophilizing protectant is weighed and dissolved in a suitable amount of water for injection to give solution 1;

B. the compound of formula (I) (preferably compound of formula (II), most preferably NL-101) or a pharmaceutically acceptable salt thereof is weighed and dissolved with a co-solvent to give a concentrated solution which is then added to the solution 1, the pH controlling agent is used to adjust the pH, and finally the volume is adjusted with water for injection to obtain diluted solution;

C. the diluted solution is filtrated, sterilized and filled in, half-stoppered, and put into a lyophilizer;

D. following lyophilization, taken out of the lyophilizer, fully stoppered and capped to give the pharmaceutical composition.

In a preferred embodiment, in step A, the lyophilizing protectant and stabilizing agent are weighed and dissolved in a suitable amount of water for injection to give solution 1;

In a preferred embodiment, the co-solvent is firstly formulated with water for injection as a solution of co-solvent. Preferably, the volume percent concentration of hydrochloric acid in the solution of the co-solvent is 30-70%, further preferably 40-60%, still further preferably 50%; the volume percent concentration of acetic acid or citric acid in the solution of the co-solvent is 30-100%, further preferably 40-80%, still further preferably 40-60%, and still further preferably 50%.

Those skilled in the art can understand that in the present invention, the pH controlling agent used to adjust the pH is preferably first formulated with water for injection, and the concentration of the pH controlling agent in the solution can be adjusted within a wide range as required.

The inventors of the present invention have found that, compared with the use of acetic acid, the use of hydrochloric acid as a co-solvent avoids the reaction of acetic acid with a compound of formula (I) (preferably a compound of formula (II), most preferably NL-101) or a pharmaceutically acceptable salt thereof to produce acetic acid (acetate) related impurities, thereby further increases the stability of the compound of formula (I) (preferably the compound of formula (II), most preferably NL-101) or a pharmaceutically acceptable salt thereof in the composition.

The beneficial effects obtained by the present invention are that, by using a chloride-containing compound as a stabilizing agent or hydrochloric acid as a co-solvent, the stability of both the diluted preparation and the lyophilized preparation of the compound of formula (I) (preferably the compound of formula (II), most preferably NL-101) or a pharmaceutically acceptable salt thereof has been significantly improved, and they can be produced stably, and meet the various demands for storage stability in storage, transportation, sales, and clinical safe use.

### DETAILED DESCRIPTION OF THE INVENTION

“Alkyl” refers to a straight or branched hydrocarbon group containing 1 to 20 carbon atoms (e.g., C₁-C₁₀). Examples of alkyl include, but are not limited to, methyl, ethyl, n-propyl, isopropyl, n-butyl, isobutyl, and tert-butyl.

“Alkenyl” refers to a straight or branched hydrocarbon group containing 2 to 20 carbon atoms (e.g., C₂-C₁₀) and one or more double bonds. Examples of alkenyl include, but are not limited to, vinyl, propenyl, and allyl.

“Alkynyl” refers to a straight or branched hydrocarbon group containing 2 to 20 carbon atoms (e.g., C₂-C₁₀) and one or more triple bonds. Examples of alkynyl include, but are not limited to, ethynyl, 1-propynyl, 1- and 2-butynyl, and 1-methyl-2-butynyl.

“Alkoxy” refers to a group formed by the attachment of an alkyl to an oxygen atom.

“Alkoxycarbonyl” refers to a group in which an alkoxy is attached to a carbonyl.

“Cycloalkyl” refers to a saturated hydrocarbon ring containing 3 to 30 carbon atoms (e.g., C₃-C₁₂). Cycloalkyl groups include, but are not limited to, cyclopropyl, cyclobutyl, cyclopentyl, cyclohexyl, cycloheptyl, and cyclooctyl.

“Heterocycloalkyl” refers to a non-aromatic 5-8 membered monocyclic, 8-12 membered bicyclic, or 11-14 membered tricyclic ring system having one or more heteroatoms (e.g., O, N, S, P or Se). Heterocycloalkyl groups include, but are not limited to, piperazinyl, pyrrolidinyl, dioxanyl, morpholinyl, and tetrahydrofuran.

“Cycloalkenyl” refers to a non-aromatic hydrocarbon ring system containing 3 to 30 carbon atoms (e.g., C₃-C₁₂) and one or more double bonds. It includes cyclopentenyl, cyclohexenyl and cycloheptenyl.

“Heterocycloalkenyl” refers to a non-aromatic 5-8 membered monocyclic, 8-12 membered bicyclic, or 11-14 membered tricyclic ring system having one or more heteroatoms (eg, O, N, S, P or Se) and one or more double bonds.

“Aryl” refers to a 6-20 membered monocyclic, bicyclic, or tricyclic aromatic ring, such as 6-carbon monocyclic, 10-carbon bicyclic, and 14-carbon tricyclic aromatic ring. Aryl groups include, but are not limited to, phenyl, naphthyl, and anthryl.

“Heteroaryl” refers to an aromatic 5-8 membered monocyclic, 8-12 membered bicyclic, or 11-14 membered tricyclic ring system having one or more heteroatoms (e.g., O, N, S, P, or Se). Heteroaryl groups include pyridyl, furyl, imidazolyl, benzimidazolyl, pyrimidinyl, thienyl, quinolinyl, indolyl, and thiazolyl.

The above-mentioned alkyl, alkenyl, alkynyl, cycloalkyl, heterocycloalkyl, cycloalkenyl, heterocycloalkenyl, alkylamino, aryl, and heteroaryl groups include both substituted and unsubstituted moieties. Possible substituents on alkylamino, cycloalkyl, heterocycloalkyl, cycloalkenyl, heterocycloalkenyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, arylamino, hydroxy, halo, oxo (O═), thio group (S═), thio, silyl, C₁-C₁₀ alkylthio, arylthio, C₁-C₁₀ alkylsulfonyl, arylsulfonyl, acylamino, aminoacyl, aminothio, amidino, mercapto, amino, thioureido, thiocyanato, sulfonamido, indolyl, ureido, cyano, nitro, acyl, thio, acyloxy, urea, carbamoyl, carboxyl, and carboxylate.

On the other hand, possible substituents on alkyl, alkenyl or alkynyl groups include all of the substituents described above except C₁-C₁₀ alkyl. Cycloalkyl, cycloalkenyl, heterocycloalkyl, heterocycloalkenyl, aryl, and heteroaryl groups can also be combined with each other.

“Amino” refers to a group having two substituents on the nitrogen, with one hydrogen or carbon atom on each substituent bonded to nitrogen via an alpha linkage. Unless otherwise indicated, the amino moiety in the compounds of the invention may contain protected amino derivatives. Suitable amino-protecting groups include acetyl, tert-butoxycarbonyl, benzyloxycarbonyl, and the like.

Halogen is selected from fluorine, chlorine, bromine or iodine.

“Haloalkyl” as a part of a lone group or large group means that “alkyl” is substituted with one or more “halo” atoms, and haloalkyl includes monohaloalkyl, dihaloalkyl, trihaloalkyl, higher haloalkyl, and the like.

“Substituted or unsubstituted” means that one substituent contains only hydrogen bonded by a covalent bond (unsubstituted) or one or more non-hydrogen substituents bonded by a covalent bond (substituted).

In the present invention, the volume percentage is calculated based on the volume of the solvent and the solution. Wherein, for the co-solvent other than hydrochloric acid, such as acetic acid or citric acid, the volume percentage is calculated based on pure acetic acid or citric acid; and the volume percentage of hydrochloric acid is calculated based on concentrated hydrochloric acid. The mass concentration of concentrated hydrochloric acid is 36%.

In the present invention, when the mass volume percentage of the stabilizing agent relates to hydrochloric acid, it is calculated based on concentrated hydrochloric acid, in which the mass concentration of concentrated hydrochloric acid is 36%.

That is, in the present invention, when the volume percentage or mass percentage of hydrochloric acid is concerned, the calculation is based on concentrated hydrochloric acid, that is, based on concentrated hydrochloric acid having a mass concentration of 36%. For example, 30% (v/v) hydrochloric acid corresponds to 30% by volume of concentrated hydrochloric acid. The present invention is not limited to the use of 36% concentrated hydrochloric acid. It will be understood by those skilled in the art that various different concentrations of hydrochloric acid can be used in the present invention, all of which are within the scope of the present invention.

The following further describes the present invention in combination with specific examples. It should be understood that these examples are only for illustrating the present invention and are not intended to limit the scope of the present invention. In addition, it should be understood that after reading the contents of the present invention, those skilled in the art can make various changes or modifications to the present invention, and these equivalent forms also fall within the scope defined by the present invention.

### EXAMPLES

### Example 1. Lyophilized Preparations for Injection Comprising a Chloride-Containing Compound as Stabilizing Agent

The chlorine-containing compound was introduced into the NL-101 lyophilized preparation for injection. The formulations 1-15, 1-1, 2-1, and 3-1 for pre-lyophilized solutions (i.e., diluted solutions) are specifically shown in Table 6 below. Wherein, formula 1 as a control does not contain chlorine ions.

TABLE 6
Formulations for chlorine-containing NL-101 lyophilized preparations for injection before lyophilization

| | Formulation | Formulation | formulation | formulation | formulation | formulation | formulation | formulation |
|---|---|---|---|---|---|---|---|---|
| | 1 | 2 | 3 | 4 | 5 | 1-1 | 2-1 | 3-1 |
| NL-101(g) | 0.25 | 0.25 | 0.25 | 0.25 | 0.25 | 0.25 | 0.25 | 0.25 |
| 50% (v/v) acetic acid | 1.25 | 2.5 | 5 | 10 | 20 | — | — | — |
| (mL) | | | | | | | | |
| 50% (v/v) citric acid | — | — | — | — | — | 1.25 | 5 | 20 |
| (mL) | | | | | | | | |
| hydroxypropyl-β- | 6.68 | 6.68 | 6.68 | 6.68 | 6.68 | 6.68 | 6.68 | 6.68 |
| cyclodextrin (g) | | | | | | | | |
| sulfobutyl ether-β- | — | — | — | — | — | — | — | — |
| cyclodextrin (g) | | | | | | | | |
| sodium chloride (g) | — | 0.22 | 0.45 | 1.5 | 2.5 | 0.45 | 0.45 | 0.45 |
| sodium bicarbonate | — | — | — | — | — | — | — | — |
| solution | | | | | | | | |
| sodium carbonate | — | — | — | — | — | — | — | — |
| solution | | | | | | | | |
| sodium hydroxide | appropriate | appropriate | appropriate | appropriate | appropriate | appropriate | appropriate | appropriate |
| solution | amount | amount | amount | amount | amount | amount | amount | amount |
| water for injection | 50 | 50 | 50 | 50 | 50 | 50 | 50 | 50 |
| to volume (mL) | | | | | | | | |
| pH | 3.0 | 4.5 | 5.0 | 5.5 | 6.0 | 5.0 | 5.0 | 5.0 |

| | formulation | formulation | formulation | formulation | formulation |
|---|---|---|---|---|---|
| | 6 | 7 | 8 | 9 | 10 |
| NL-101(g) | 0.25 | 0.25 | 0.25 | 0.25 | 0.25 |
| 50% (v/v) acetic acid | 1.25 | 1.25 | 1.25 | 1.25 | 1.25 |
| (mL) | | | | | |
| hydroxypropyl-β- | 6.68 | 2.50 | 12.50 | 6.68 | 6.68 |
| cyclodextrin (g) | | | | | |
| sulfobutyl ether-β- | — | — | — | — | — |
| cyclodextrin (g) | | | | | |
| sodium chloride (g) | 5 | 1.5 | 1.5 | 1.5 | 1.5 |
| sodium bicarbonate | — | — | — | appropriate | — |
| solution | | | | amount | |
| sodium carbonate | — | — | — | — | appropriate |
| solution | | | | | amount |
| sodium hydroxide | appropriate | appropriate | appropriate | — | — |
| solution | amount | amount | amount | | |
| water for injection | 50 | 50 | 50 | 50 | 50 |
| to volume (mL) | | | | | |
| pH | 6.5 | 7.0 | 7.0 | 3.0 | 7.0 |
| | formulation | formulation | formulation | formulation | formulation |
| | 11 | 12 | 13 | 14 | 15 |
| NL-101(g) | 0.25 | 0.25 | 0.25 | 0.25 | 0.25 |
| 50% (v/v) acetic acid | 1.25 | 1.25 | 1.25 | 2.5 | 5 |
| (mL) | | | | | |
| hydroxypropyl-β- | — | — | — | — | — |
| cyclodextrin (g) | | | | | |
| sulfobutyl ether-β- | 2.50 | 6.68 | 12.50 | 6.68 | 6.68 |
| cyclodextrin (g) | | | | | |
| sodium chloride (g) | 5 | 1.5 | 1.5 | 1.5 | 1.5 |
| sodium bicarbonate | — | — | — | appropriate | — |
| solution | | | | amount | |
| sodium carbonate | — | — | — | — | appropriate |
| solution | | | | | amount |
| sodium hydroxide | appropriate | appropriate | appropriate | — | — |
| solution | amount | amount | amount | | |
| water for injection | 50 | 50 | 50 | 50 | 50 |
| to volume (mL) | | | | | |
| pH | 3.0 | 4.0 | 5.0 | 6.0 | 7.0 |

Lyophilized preparations for injection were prepared by the following method:

A. hydroxypropyl-β-cyclodextrin or sulfobutyl ether-β-cyclodextrin and sodium chloride or potassium chloride were weighed in prescribed amounts and dissolved in a suitable amount of water for injection to give solution 1;

B. NL-101 was weighed in prescribed amount and dissolved with 50% solution of acetic acid or 50% solution of citric acid in prescribed amounts to obtain a concentrated solution which was then added to the solution 1, well stirred, and 40% (w/v) sodium hydroxide, sodium carbonate or sodium bicarbonate solution were used for adjusting pH, and finally the volume was adjusted with water for injection to obtain diluted solution;

C. the diluted solution was filtrated, sterilized and filled in, half-stoppered, and put into a lyophilizer;

D. following lyophilization, taken out of the lyophilizer, fully stoppered and capped to give the lyophilized preparations for injection.

The diluted solutions prepared according to the formulations 1-15, 1-1, 2-1, 3-1 and according to Example 2 of CN103826630A were left to stand for 2 hours at room temperature (25±2° C.) and the stability of each diluted solution was tested by high performance liquid chromatography. The results are shown in Table 7 below.

TABLE 7
Stability test results for formulations 1-15, 1-1, 2-1, 3-1 diluted solutions after placed at room temperature (25 ± 2° C.) for 2 hours

| | formulation | | | | | | | | |
|---|---|---|---|---|---|---|---|---|---|
| | CN103826630A | formulation | formulation | formulation | formulation | formulation | formulation | formulation | formulation |
| | Example 2 | 1 | 2 | 3 | 4 | 5 | 1-1 | 2-1 | 3-1 |
| appearance | clear | clear | clear | clear | clear | clear | clear | clear | clear |
| purity | 88.06% | 88.22% | 91.06% | 97.65% | 97.72% | 97.68% | 97.23% | 97.55% | 97.69% |
| the largest | 6.70% | 6.79% | 4.51% | 1.83% | 1.25% | 1.98% | 1.78% | 1.87% | 1.90% |
| single | | | | | | | | | |
| miscellaneous | | | | | | | | | |

| | formulation | | | | | |
|---|---|---|---|---|---|---|
| | CN103826630A | formulation | formulation | formulation | formulation | formulation |
| | Example 2 | 6 | 7 | 8 | 9 | 10 |
| appearance | clear | a small | clear | clear | clear | clear |
| | | amount of | | | | |
| | | solid | | | | |
| | | precipitated | | | | |
| purity | 88.06% | 96.68% | 97.06% | 97.56% | 97.42% | 97.61% |
| the largest | 6.70% | 1.28% | 2.51% | 1.43% | 1.35% | 1.38% |
| single | | | | | | |
| miscellaneous | | | | | | |
| | formulation | | | | | |
| | CN103826630A | formulation | formulation | formulation | formulation | formulation |
| | Example 2 | 11 | 12 | 13 | 14 | 15 |
| appearance | clear | a small | clear | clear | clear | clear |
| | | amount of | | | | |
| | | solid | | | | |
| | | precipitated | | | | |
| purity | 88.06% | 96.02% | 97.77% | 97.65% | 97.01% | 97.68% |
| the largest | 6.70% | 2.88% | 1.50% | 1.31% | 1.55% | 1.61% |
| single | | | | | | |
| miscellaneous | | | | | | |

The results showed that the addition of chlorine-containing compounds to the formulations 2-15, 1-1, 2-1, and 3-1 significantly increased the stability of the diluted solution, and the greater the concentration, the better the effect of increasing the stability. However, the addition of sodium chloride or potassium chloride had an adverse effect on salting-out of NL-101 dissolution (decreasing the solubility of NL-101 in the solution), for example, solid precipitation in formulations 6 and 11. The use of hydroxypropyl-β-cyclodextrin or sulfobutyl ether-β-cyclodextrin had no effect on the stability of NL-101; however, there was a certain correlation between the content of hydroxypropyl-β-cyclodextrin or sulfobutyl ether-β-cyclodextrin and stability. The pH was adjusted with sodium bicarbonate, sodium carbonate or sodium hydroxide solution, which had little effect on the stability of NL-101 in the range of pH 3.0-7.0.

The NL-101 lyophilized preparation for injection prepared according to the formulation 4 was taken and subjected to an accelerated test at 40° C., and the stability was tested by high performance liquid chromatography. It was found that the lyophilized preparation obtained by formulation 4 was significantly more stable than the lyophilized preparation prepared in Example 2 of CN103826630A. The results are shown in Table 8 below.

TABLE 8
Stability test results for formulation 4 lyophilized
preparation in accelerated test at 40° C.

| | Day 0 | Day 5 |
|---|---|---|
| purity | 97.72% | 89.11% |

From the above table, it can be seen that the NL-101 lyophilized preparation prepared according to the formulation 4 was obviously more stable than that of Example 2 of CN103826630A, but after being accelerated for 5 days at 40° C., nearly 10% of NL-101 had been degraded as compared with day 0. Accelerated test samples were further detected by high performance liquid chromatography in connection with mass spectrometry, and the main degradation product was found to be the product of a chemical reaction between acetate and NL-101. The results are shown in Table 9 below.

TABLE 9
Results for formulation 4 lyophilized preparation
in accelerated test at 40° C.

| | Day 0 | Day 5 |
|---|---|---|
| Content of impurities related to acetic acid (acetate) | 1.25% | 8.42% |

### Example 2. Lyophilized Preparations for Injection Comprising Hydrochloric Acid as Co-Solvent

Through the study of formulations 1-15, the inventors found that acetic acid (acetate) and active pharmaceutical ingredient NL-101 will undergo a chemical reaction, but the introduction of chlorine ions is very helpful for improving the stability of the preparation (whether it is a diluted solution or a lyophilized preparation). Since NL-101 is extremely unstable in strong acids, although hydrochloric acid can dissolve NL-101, it is theoretically not the preferred solvent for NL-101 preparations. In order to further improve the stability of the NL-101 preparation, the inventors have found through a large number of experiments that hydrochloric acid can be used instead of acetic acid. On the one hand, no acetate ions can be introduced; on the other hand, hydrochloric acid is neutralized with pH controlling agent including sodium bicarbonate, sodium carbonate, or sodium hydroxide to form sodium chloride. Chloride can increase the stability of NL-101 in the preparation, thereby simplifying the formulation.

The NL-101 preparations were prepared using hydrochloric acid as a co-solvent and the solution formulation 16-29 before lyophilization is shown in Table 10 below.

TABLE 10
Formulations for NL-101 lyophilized preparations for injection
using hydrochloric acid as a co-solvent before lyophilization

| | formulation 16 | formulation 17 | formulation 18 | formulation 19 | formulation 20 |
|---|---|---|---|---|---|
| NL-101(g) | 0.25 | 0.25 | 0.25 | 0.25 | 0.25 |
| 30% (v/v) hydrochloric acid (mL) | — | — | 4.2 | — | 5 |
| 50% (v/v) hydrochloric acid (mL) | 2.5 | 5 | — | — | — |
| 70% (v/v) hydrochloric acid (mL) | — | — | — | 10 | — |
| hydroxypropyl-β-cyclodextrin (g) | 6.68 | — | 6.68 | 6.68 | — |
| sulfobutyl ether-β-cyclodextrin (g) | — | 6.68 | — | — | 6.68 |
| sodium chloride (g) | 0.5 | 1.0 | — | 1.0 | — |
| sodium bicarbonate solution | — | — | — | — | — |
| sodium carbonate solution | — | — | — | — | — |
| sodium hydroxide solution | appropriate | appropriate | appropriate | appropriate | appropriate |
| | amount | amount | amount | amount | amount |
| water for injection to volume (mL) | 50 | 50 | 50 | 50 | 50 |
| pH | 3.0 | 3.0 | 5.0 | 5.0 | 6.0 |
| | formulation 21 | formulation 22 | formulation 23 | formulation 24 | formulation 25 |
| NL-101(g) | 0.25 | 0.25 | 0.25 | 0.25 | 0.25 |
| 30% (v/v) hydrochloric acid (mL) | — | — | — | — | — |
| 50% (v/v) hydrochloric acid (mL) | 2.5 | 2.5 | 2.5 | 2.5 | 2.5 |
| 70% (v/v) hydrochloric acid (mL) | — | — | — | — | — |
| hydroxypropyl-β-cyclodextrin (g) | 6.68 | 6.68 | 6.68 | — | — |
| sulfobutyl ether-β-cyclodextrin (g) | — | — | — | 6.68 | 6.68 |
| sodium chloride (g) | 1.0 | — | 0.5 | 0.5 | — |
| sodium bicarbonate solution | appropriate | — | — | appropriate | — |
| | amount | | | amount | |
| sodium carbonate solution | — | — | appropriate | — | appropriate |
| | | | amount | | amount |
| sodium hydroxide solution | — | appropriate | — | — | — |
| | | amount | | | |
| water for injection to volume (mL) | 50 | 50 | 50 | 50 | 50 |
| pH | 4.0 | 5.0 | 5.0 | 6.0 | 7.0 |

| | formulation 26 | formulation 27 | formulation 28 | formulation 29 |
|---|---|---|---|---|
| NL-101(g) | 0.25 | 0.25 | 0.25 | 0.25 |
| 30% (v/v) hydrochloric acid (mL) | — | — | — | — |
| 50% (v/v) hydrochloric acid (mL) | 2.5 | 10 | 10 | 2.5 |
| 70% (v/v) hydrochloric acid (mL) | — | — | — | — |
| hydroxypropyl-β-cyclodextrin (g) | 2.5 | 12.5 | — | — |
| sulfobutyl ether-β-cyclodextrin (g) | — | — | 2.5 | 12.5 |
| sodium chloride (g) | — | — | — | — |
| sodium bicarbonate solution | — | — | appropriate | — |
| | | | amount | |
| sodium carbonate solution | — | — | — | — |
| sodium hydroxide solution | appropriate | appropriate | — | appropriate |
| | amount | amount | | amount |
| water for injection to volume (mL) | 50 | 50 | 50 | 50 |
| pH | 4.0 | 7.0 | 5.0 | 6.0 |

Lyophilized preparations for injection were prepared by the following method:

A. hydroxypropyl-β-cyclodextrin or sulfobutyl ether-β-cyclodextrin were weighed in prescribed amounts, and, if any, sodium chloride was weighed in prescribed amount, and dissolved in a suitable amount of water for injection to give solution 1;

B. NL-101 was weighed in prescribed amount and dissolved with hydrochloric acid in prescribed amounts to obtain a concentrated solution which was then added to the solution 1, well stirred, and sodium bicarbonate, sodium carbonate or sodium hydroxide solution were used for adjusting pH, and finally the volume was adjusted with water for injection to obtain diluted solution;

C. the diluted solution was filtrated, sterilized and filled in, half-stoppered, and put into a lyophilizer;

D. following lyophilization, taken out of the lyophilizer, fully stoppered and capped to give the lyophilized preparations for injection.

The diluted solutions prepared according to the formulations 16-29 and according to Example 2 of CN103826630A were left to stand for 2 hours at room temperature (25±2° C.) and the stability of each diluted solution was tested by high performance liquid chromatography. The results are shown in Table 11 below.

TABLE 11
Stability test results for formulations 16-29 diluted solutions after
placed at room temperature (25 ± 2° C.) for 2 hours

| | formulation | | | | | |
|---|---|---|---|---|---|---|
| | CN103826630A | formulation | formulation | formulation | formulation | formulation |
| | Example2 | 16 | 17 | 18 | 19 | 20 |
| appearance | clear | clear | clear | clear | clear | clear |
| purity | 88.06% | 98.30% | 98.21% | 98.18% | 98.33% | 98.20% |
| the largest | 6.70% | 0.77% | 0.89% | 1.07% | 0.94% | 1.11% |
| single | | | | | | |
| miscellaneous | | | | | | |
| | formulation | | | | | |
| | CN103826630A | formulation | formulation | formulation | formulation | formulation |
| | Example 2 | 21 | 22 | 23 | 24 | 25 |
| appearance | clear | clear | clear | clear | clear | clear |
| purity | 88.06% | 98.23% | 98.30% | 98.25% | 98.42% | 98.07% |
| the largest | 6.70% | 0.78% | 0.91% | 0.88% | 0.83% | 0.93% |
| single | | | | | | |
| miscellaneous | | | | | | |

| | formulation | | | | |
|---|---|---|---|---|---|
| | CN103826630A | formulation | formulation | formulation | formulation |
| | Example 2 | 26 | 27 | 28 | 29 |
| appearance | clear | clear | clear | clear | clear |
| purity | 88.06% | 97.24% | 98.31% | 97.43% | 98.19% |
| the largest | 6.70% | 1.37% | 0.84% | 1.31% | 0.75% |
| single | | | | | |
| miscellaneous | | | | | |

The results showed that the use of hydrochloric acid as a solvent significantly increased the stability of the NL-101 diluted solution. The concentration of hydrochloric acid is related to the ability to dissolve NL-101, but the concentration of hydrochloric acid has little effect on the stability of diluted solutions. The use of hydroxypropyl-β-cyclodextrin or sulfobutyl ether-β-cyclodextrin had no effect on the stability of NL-101; however, there was a certain correlation between the content of hydroxypropyl-β-cyclodextrin or sulfobutyl ether-β-cyclodextrin and stability. Formulations 26 and 28 were the most unstable. The pH was adjusted with sodium bicarbonate, sodium carbonate or sodium hydroxide solution, which had no effect on the stability of NL-101. Due to the reaction between hydrochloric acid in a prescribed amount and pH controlling agent to produce sodium chloride, whether sodium chloride was added or not had no significant effect on the stability of NL-101. In the pH range of 3.0-7.0 for diluted solutions, the pH value had no significant effect on the stability of NL-101.

The NL-101 lyophilized preparation for injection prepared according to the formulations 16 and 22 were taken and subjected to an accelerated test at 40° C., and the accelerated test samples were test by high performance liquid chromatography in connection with mass spectrometry. The results are shown in Table 12 below.

TABLE 12
Stability test results for lyophilized preparations of formulations 16 and 22 in accelerated test at 40° C.

| | Day 0 | | | Day 5 | | |
|---|---|---|---|---|---|---|
| | | The largest | Content of impurities | | The largest | Content of impurities |
| | | single | related to acetic | | single | related to acetic |
| Test item | Purity | miscellaneous | acid (acetate) | Purity | miscellaneous | acid (acetate) |
| formulation 16 | 98.30% | 0.77% | 0 | 98.27% | 0.78% | 0 |
| formulation 22 | 98.23% | 0.93% | 0 | 98.15% | 0.97% | 0 |

The results showed that the lyophilized preparations prepared according to formulations 16 and 22 were fairly stable. In the accelerated test at 40° C. for 5 days, the purity of NL-101 was almost not reduced, and there was no increase in the related substances.

### Example 3

In order to further verify whether the formulation and the process of the present invention can achieve industrialized large-scale production, and whether the NL-101 lyophilized preparation of the present invention is stable enough to meet the needs of storage, sale and clinical use, based on formulation 22 and its preparation process, the scaled-up pilot scale production was carried out on the industrialized production line, 3 batches were continuously produced, and the lyophilized preparations were tested for stability in additional experiment at 40° C. for 6 months.

Taking the formulation of the formulation 22 as an example, the pilot scale is enlarged in three batches, and the formulation of the solution before lyophilization is shown in Table 13 below.

TABLE 13
Formulations for NL-101 lyophilized preparations for injection
for three batches in pilot scale up production

| | Batch | | |
|---|---|---|---|
| Composition | Batch 1 | Batch 2 | Batch 3 |
| NL-101(g) | 150 | 150 | 150 |
| 50% (v/v) hydrochloric | 1500 | 1500 | 1500 |
| acid (mL) | | | |
| hydroxypropyl-β- | 4008 | 4008 | 4008 |
| cyclodextrin (g) | | | |
| sodium hydroxide | appropriate | appropriate | appropriate |
| solution | amount | amount | amount |
| water for injection | 30000 | 30000 | 30000 |
| to volume (mL) | | | |
| pH | 5.0 | 5.0 | 5.0 |

Lyophilized preparations for injection were prepared by the following method:

A. hydroxypropyl-β-cyclodextrin was weighed in prescribed amounts and dissolved in a suitable amount of water for injection to give solution 1;

B. NL-101 was weighed in prescribed amount and dissolved with hydrochloric acid in prescribed amounts in an ice bath to obtain a concentrated solution which was then added to the solution 1, well stirred, and sodium hydroxide solution were used for adjusting pH, and finally the volume was adjusted with water for injection to obtain diluted solution;

C. the diluted solution was filtrated, sterilized and filled in on-line, half-stoppered, and put into a lyophilizer on-line;

D. after all the samples were put into the lyophilizer, the lyophilizer was closed and began to perform lyophilization;

E. following lyophilization, taken out of the lyophilizer on-line, fully stoppered and capped on-line;

F. appearance was inspected, and the samples with qualified appearance were placed in the intermediate turnover chamber at 4-8° C. for storage and submitted for inspection.

G. after passing inspection, being labeled and packaged to obtain the finished products.

Three batches of diluted solutions were taken and placed at room temperature (25±2° C.) for different periods of time. The stability of each diluted solution was tested by high performance liquid chromatography. The results are shown in Table 14 below.

TABLE 14
Stability test results for three batches of diluted solutions after placed at
room temperature (25 ± 2° C.) for different periods of time

| Sample | Test item | 0 h | 2 h | 4 h | 6 h |
|---|---|---|---|---|---|
| Batch 1 | purity | 98.48% | 98.40% | 98.28% | 98.08% |
| | total | 1.41% | 1.40% | 1.42% | 1.42% |
| | impurity | | | | |
| Batch 2 | purity | 98.58% | 98.49% | 98.39% | 98.28% |
| | total | 1.26% | 1.27% | 1.27% | 1.27% |
| | impurity | | | | |
| Batch 3 | purity | 98.39% | 98.30% | 98.21% | 98.06% |
| | total | 1.51% | 1.51% | 1.52% | 1.53% |
| | impurity | | | | |

The results showed that the three batches of diluted solutions were stable at room temperature (25±2° C.) for 6 hours.

The above three batches of lyophilized powder for injection were taken and subjected to accelerated experiments at 40° C. The contents and total impurities were measured at 0, 1, 3, and 6 months, respectively. The experimental data is shown in Table 15 below.

TABLE 15
Result for three batches of lyophilized powder for injection in the
accelerated experiment at 40° C. for different periods of time

| | | Accel- | Accel- | Accel- | Accel- |
|---|---|---|---|---|---|
| | | erated | erated | erated | erated |
| | | for 0 | for 1 | for 3 | for 6 |
| Sample | Test item | month | month | months | months |
| Batch 1 | content | 98.38% | 98.20% | 97.88% | 97.28% |
| | total impurity | 1.43% | 1.44% | 1.44% | 1.45% |
| Batch 2 | content | 98.40% | 98.05% | 97.78% | 97.32% |
| | total impurity | 1.27% | 1.27% | 1.28% | 1.29% |
| Batch 3 | content | 98.02% | 97.81% | 97.42% | 97.05% |
| | total impurity | 1.54% | 1.54% | 1.55% | 1.57% |

The results showed that the content and impurities of the NL-101 lyophilized preparation for injection prepared according to the formulation and process of the present invention can be controlled within the acceptable range under the conditions of acceleration at 40° C. for 6 months. According to the accelerated test results at 40° C., it is presumed that under the condition of storage, transportation and sales at room temperature (25±2° C.), it can remain stable for at least 2 years and fully meet the needs of clinical use.

The embodiments of the present invention have been described above. However, the present invention is not limited to the above embodiment. Any modification, equivalent replacement, and improvement made within the spirit and principle of the present invention shall fall within the protection scope of the present invention.

1. A pharmaceutical composition, characterized in comprising a compound of formula (I) or a pharmaceutically acceptable salt thereof and a stabilizing agent, wherein the stabilizing agent is a pharmaceutically acceptable chloride-containing compound, 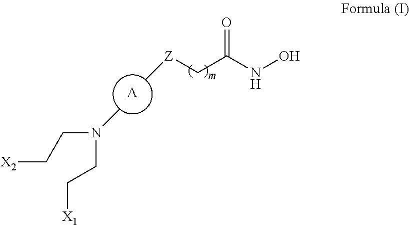
wherein,
m is an integer selected from 5-16;
Z is absent or selected from C(RaRb), O, S, C(O), N(Ra), SO₂, OC(O), C(O)O, OSO₂, S(O₂)O, C(O)S, SC(O), C(O)C(O), C(O)N(Ra), N(Ra)C(O), S(O₂)N(Ra), N(Ra)S(O₂), OC(O)N(Ra), N(Ra)C(O)O, N(Ra)C(O)S, or N(Ra)C(O)N(Rb), wherein Ra and Rb are each independently H, alkyl, alkenyl or alkynyl;
X₁ and X₂ are each independently halogen or OSO₂Rc, wherein Rc is alkyl, alkenyl or alkynyl;
Q is selected from cycloalkyl, heterocycloalkyl, cycloalkenyl, heterocycloalkenyl, aryl or heteroaryl, optionally substituted with one or more substituents selected from alkyl, alkenyl, alkynyl, cycloalkyl, heterocycloalkyl, cycloalkenyl, heterocycloalkenyl, aryl, heteroaryl, halogen, nitro, oxo, —C═NH, cyano, alkyl-Rd, ORd, OC(O)Rd, OC(O)ORd, OC(O)SRd, SRd, C(O)Rd, C(O)ORd, C(O)SRd, C(O)NReRf, SORd, SO₂Rd, NReRf, or N(Re)C(O)Rf, wherein Rd, Re, and Rf are each independently selected from H, alkyl, alkenyl, alkynyl, cycloalkyl, heterocycloalkyl, aryl, heteroaryl, halogen, cyano, amine, nitro, hydroxyl, or alkoxy.

2. The pharmaceutical composition according to claim 1, characterized in that
m is selected from 5, 6, 7 or 8;
Z is absent or selected from CH₂, O, CO, NH, SO₂, OC(O), C(O)O, C(O)S, NHC(O), C(O)NH, OC(O)NH, NHC(O)O, or NHC(O)S;
X₁ and X₂ are independently halogen; and
Q is 9-10 membered aryl or heteroaryl;
more preferably, Z is absent or selected from CH₂, O, CO, NH, SO₂, NHC(O), or C(O)NH,
still more preferably, the compound of formula (I) is represented by the following formula (II): 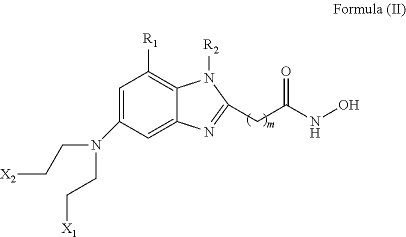
wherein R₁ and R₂ are each independently selected from H, alkyl, alkenyl, alkynyl, cycloalkyl, heterocycloalkyl, halogen, —C═NH, amine, cyano, hydroxy, or alkoxy.

3. The pharmaceutical composition according to claim 1, characterized in comprising NL-101 or a pharmaceutically acceptable salt thereof and a stabilizing agent, wherein the stabilizing agent is a pharmaceutically acceptable chloride-containing compound.

4. The pharmaceutical composition according to claim 1, characterized in further comprising a co-solvent, a lyophilizing protectant, a pH controlling agent, and water for injection;
preferably, the stabilizing agent is a mixture selected from one or more of sodium chloride, potassium chloride, and hydrochloric acid, and further preferably is sodium chloride;
preferably, the co-solvent is a pharmaceutically acceptable acidic solvent, further preferably acetic acid and/or citric acid;
preferably, the lyophilizing protectant is a pharmaceutically acceptable cyclic polysaccharide or a mixture thereof, preferably the cyclic polysaccharide is cyclodextrin, cyclomannin, cycloaltrin, cyclofructin or an analog thereof, further preferably the cyclic polysaccharide is cyclodextrin or a derivative thereof, further preferably the cyclic polysaccharide is α-cyclodextrin or a derivative thereof, β-cyclodextrin or a derivative thereof, or γ-cyclodextrin or a derivative thereof, further preferably the cyclic polysaccharide is β-cyclodextrin or a derivative thereof, most preferably the cyclic polysaccharide is β-cyclodextrin, sulfobutylether-β-cyclodextrin and/or hydroxypropyl-β-cyclodextrin;
preferably, the pH controlling agent is a mixture selected from one or more of sodium hydroxide, sodium carbonate, potassium carbonate, sodium hydrogencarbonate, potassium hydrogencarbonate, sodium citrate, and potassium citrate, and further preferably is sodium hydroxide.

5. The pharmaceutical composition according to claim 1, characterized in that the compound of the formula (I) or the compound of the formula (II) or NL-101 or a pharmaceutically acceptable salt thereof has a mass volume percentage concentration of 0.1-5.0%, preferably 0.2-2.0%, further preferably 0.5-1.0%, still further preferably 0.5%;
preferably, the stabilizing agent has a mass volume percentage concentration of 0.4-10.0%, further preferably 0.9-8.0%, still further preferably 1.0-7.0%, still further preferably 2.0-6.0%, still further preferably 3.0-5.0%;
preferably, the co-solvent has a mass volume percentage concentration of 0.5-25.0%, further preferably 1.0-20.0%, still further preferably 1.0-10.0%, still further preferably 1.25-5.0%;
preferably, the lyophilizing protectant has a mass volume percentage concentration of 2.0-35.0%, further preferably 5.0-30.0%, still further preferably 10.0-20.0%;
preferably, the pH of the composition is 3.0-7.0, further preferably 4.0-6.0, still further preferably 5.0.

6. A lyophilized pharmaceutical composition for injection, characterized in that the lyophilized pharmaceutical composition for injection is prepared by lyophilizing the pharmaceutical composition according to claim 1.

7. A method for preparing a pharmaceutical composition according to claim 6, characterized in comprising the steps of:
A. the lyophilizing protectant and stabilizing agent are weighed and dissolved in a suitable amount of water for injection to give solution 1;
B. the compound of formula (I) (preferably compound of formula (II), most preferably NL-101) or a pharmaceutically acceptable salt thereof is weighed and dissolved with a co-solvent to give a concentrated solution which is then added to the solution 1, the pH controlling agent is used to adjust the pH, and finally the volume is adjusted with water for injection to obtain diluted solution;
C. the diluted solution is filtrated, sterilized and filled in, half-stoppered, and put into a lyophilizer;
D. following lyophilization, taken out of the lyophilizer, fully stoppered and capped to give the pharmaceutical composition;
preferably, the co-solvent is firstly formulated with water for injection as a solution of co-solvent;
further preferably, the co-solvent is acetic acid or citric acid, and the volume percent concentration of acetic acid or citric acid in the solution of the co-solvent is 30-100%, further preferably 40-80%, still further preferably 40-60%, and still further preferably 50%.

8. A pharmaceutical composition, characterized in comprising a compound of formula (I) or a pharmaceutically acceptable salt thereof and a co-solvent, the co-solvent is hydrochloric acid, or a mixture of hydrochloric acid with a pharmaceutically acceptable acidic solvent; preferably, the co-solvent is hydrochloric acid, or a mixture of hydrochloric acid with acetic acid and/or citric acid; 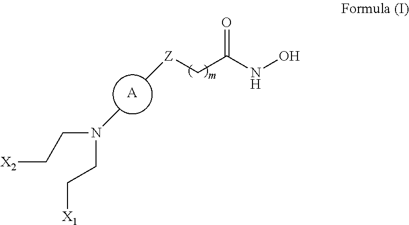
wherein,
m is an integer selected from 5-16;
Z is absent or selected from C(RaRb), O, S, C(O), N(Ra), SO₂, OC(O), C(O)O, OSO₂, S(O₂)O, C(O)S, SC(O), C(O)C(O), C(O)N(Ra), N(Ra)C(O), S(O₂)N(Ra), N(Ra)S(O₂), OC(O)N(Ra), N(Ra)C(O)O, N(Ra)C(O)S or N(Ra)C(O)N(Rb), wherein Ra and Rb are each independently H, alkyl, alkenyl or alkynyl;
X₁ and X₂ are each independently halogen or OSO₂Rc, wherein Rc is alkyl, alkenyl or alkynyl;
Q is selected from cycloalkyl, heterocycloalkyl, cycloalkenyl, heterocycloalkenyl, aryl or heteroaryl, optionally substituted with one or more substituents selected from alkyl, alkenyl, alkynyl, cycloalkyl, heterocycloalkyl, cycloalkenyl, heterocycloalkenyl, aryl, heteroaryl, halogen, nitro, oxo, —C═NH, cyano, alkyl-Rd, ORd, OC(O)Rd, OC(O)ORd, OC(O)SRd, SRd, C(O)Rd, C(O)ORd, C(O)SRd, C(O)NReRf, SORd, SO₂Rd, NReRf, or N(Re)C(O)Rf, wherein Rd, Re, and Rf are each independently selected from H, alkyl, alkenyl, alkynyl, cycloalkyl, heterocycloalkyl, aryl, heteroaryl, halogen, cyano, amine, nitro, hydroxyl, or alkoxy;
preferably,
m is selected from 5, 6, 7 or 8;
Z is absent or selected from CH₂, O, CO, NH, SO₂, OC(O), C(O)O, C(O)S, NHC(O), C(O)NH, OC(O)NH, NHC(O)O, or NHC(O)S;
X₁ and X₂ are independently halogen; and
Q is 9-10 membered aryl or heteroaryl;
more preferably, Z is absent or selected from CH₂, O, CO, NH, SO₂, NHC(O), or C(O)NH;
still more preferably, the compound of formula (I) is represented by the following formula (II): 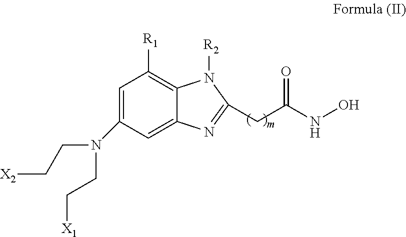
wherein R₁ and R₂ are each independently selected from H, alkyl, alkenyl, alkynyl, cycloalkyl, heterocycloalkyl, halogen, —C═NH, amine, cyano, hydroxy, or alkoxy;
most preferably, the compound of formula (I) is selected from NL-101.

9. The pharmaceutical composition according to claim 8, characterized in further comprising a lyophilizing protectant, a pH controlling agent, and water for injection;
preferably, the lyophilizing protectant is a pharmaceutically acceptable cyclic polysaccharide or a mixture thereof, preferably the cyclic polysaccharide is cyclodextrin, cyclomannin, cycloaltrin, cyclofructin or an analog thereof, further preferably the cyclic polysaccharide is cyclodextrin or a derivative thereof, further preferably the cyclic polysaccharide is α-cyclodextrin or a derivative thereof, β-cyclodextrin or a derivative thereof, or γ-cyclodextrin or a derivative thereof, further preferably the cyclic polysaccharide is β-cyclodextrin or a derivative thereof, most preferably the cyclic polysaccharide is β-cyclodextrin, sulfobutylether-β-cyclodextrin and/or hydroxypropyl-β-cyclodextrin;
preferably, the pH controlling agent is a mixture selected from one or more of sodium hydroxide, sodium carbonate, potassium carbonate, sodium hydrogencarbonate, potassium hydrogencarbonate, sodium citrate, and potassium citrate, and further preferably is sodium hydroxide;
preferably, the pharmaceutical composition further comprises a stabilizing agent, further preferably, the stabilizing agent is a mixture selected from one or more of sodium chloride, potassium chloride, and hydrochloric acid, and still further preferably is sodium chloride.

10. The pharmaceutical composition according to claim 8, characterized in that the compound of the formula (I) or the compound of the formula (II) or NL-101 or a pharmaceutically acceptable salt thereof has a mass volume percentage concentration of 0.1-5.0%, preferably 0.2-2.0%, further preferably 0.5-1.0%, still further preferably 0.5%;
preferably, the co-solvent has a mass volume percentage concentration of 0.5-25.0%, further preferably 1.0-20.0%, still further preferably 1.0-10.0%, still further preferably 1.25-5.0%;
preferably, the lyophilizing protectant has a mass volume percentage concentration of 2.0-35.0%, further preferably 5.0-30.0%, still further preferably 10.0-20.0%;
preferably, the stabilizing agent has a mass volume percentage concentration of 0-10.0%, further preferably 0.9-8.0%, still further preferably 1.0-7.0%, still further preferably 2.0-6.0%, still further preferably 3.0-5.0%;
preferably, the pH of the composition is 3.0-7.0, further preferably 4.0-6.0, still further preferably 5.0.

11. A lyophilized pharmaceutical composition for injection, characterized in that the lyophilized pharmaceutical composition for injection is prepared by lyophilizing the pharmaceutical composition according to claim 8.

12. A method for preparing a pharmaceutical composition according to claim 11, characterized in comprising the steps of:
A. the lyophilizing protectant is weighed and dissolved in a suitable amount of water for injection to give solution 1;
B. the compound of formula (I) (preferably compound of formula (II), most preferably NL-101) or a pharmaceutically acceptable salt thereof is weighed and dissolved with a co-solvent to give a concentrated solution which is then added to the solution 1, the pH controlling agent is used to adjust the pH, and finally the volume is adjusted with water for injection to obtain diluted solution;
C. the diluted solution is filtrated, sterilized and filled in, half-stoppered, and put into a lyophilizer;
D. following lyophilization, taken out of the lyophilizer, fully stoppered and capped to give the pharmaceutical composition;
preferably, in step A, the lyophilizing protectant and stabilizing agent are weighed and dissolved in a suitable amount of water for injection to give solution 1;
preferably, the co-solvent is firstly formulated with water for injection as a solution of co-solvent;
further preferably, the volume percent concentration of hydrochloric acid in the solution of the co-solvent is 30-70%, still further preferably 40-60%, still further preferably 50%;
further preferably, the volume percent concentration of acetic acid or citric acid in the solution of the co-solvent is 30-100%, further preferably 40-80%, still further preferably 40-60%, and still further preferably 50%.
